(19) United States
(12) Patent Application Publication
(10) Pub. No.: US 2025/0154453 A1
(43) Pub. Date: May 15, 2025

(54) METHOD FOR PRODUCING TRANSPLANT MATERIAL, AND TRANSPLANT MATERIAL

(71) Applicant: PorMedTec Co., Ltd., Kawasaki-shi, Kanagawa (JP)

(72) Inventors: Hiroshi NAGASHIMA, Kawasaki-shi, Kanagawa (JP); Hitomi MATSUNARI

(21) Appl. No.: 18/725,479

(22) PCT Filed: Dec. 27, 2022
(86) PCT No.: PCT/JP2022/048145

§ 371 Date: Jun. 28, 2024

(30) Foreign Application Priority Data
Dec. 28, 2021 (JP) 2021-215284
Sep. 29, 2022 (JP) 2022-156136

(51) Int. Cl. C12N 5/07; A61L 27/38; A61L 27/40; A61L 27/52; C08L 5/04; C12N 5/00

(52) U.S. Cl. CPC C12N 5/06; A61L 27/38; A61L 27/40; A61L 27/52; C08L 5/04; C12N 5/562

(57) ABSTRACT

Provided is a method for producing a transplant material that allows a foreign substance to be injected easily into an organ removed from an animal body. Also provided is a transplant material that undergoes reduced damage despite a foreign substance being injected. The present invention is a method for producing a transplant material containing an organ into which a foreign substance is injected, the method including a step for bringing at least a portion of an organ removed from an animal body into contact with a plastic member to carry out positioning, and a step for injecting a foreign substance into the positioned organ.

### CROSS REFERENCE TO RELATED APPLICATIONS

This is the U.S. national stage of application No. PCT/JP2022/048145, filed on Dec. 27, 2022. Priority under 35 U.S.C. § 119(a) and 35 U.S.C. § 365(b) is claimed from Japanese Application No. 2021-215284, filed Dec. 28, 2021, and from Japanese Application No. 2022-156136, filed Sep. 29, 2022, the disclosure of which is also incorporated herein by reference.

### TECHNICAL FIELD

The present invention relates to a method for producing a transplant material containing an organ into which a foreign substance is injected, which is suitable as a product for regenerative medicine, and a transplant material. In particular, the present invention relates to a method for producing a material for xenotransplantation containing an organ into which a foreign substance is injected, and a material for xenotransplantation.

### BACKGROUND ART

Organ regeneration is actively being developed for various organs. For example, regarding kidneys which have been considered to be complicated and difficult from the perspective of organ regeneration, an “embryonic organ complementation”, by which a foreign substance such as human mesenchymal stem cells is injected into a kidney development zone of an animal in a developmental stage, and a heteroecious developmental program is used, has been developed (for example, see Non-Patent Document 1).

Regarding the embryonic organ complementation, for example, when a foreign substance (for example, human mesenchymal stem cells) is injected into an organ (such as a metanephros) of an animal body (such as a fetus body), puncturing by a needle-shaped member such as a glass capillary tube is difficult in a state in which the organ such as the metanephros is removed from the fetus body. Therefore, in a state in which the animal body (such as a fetus body) is incised to some extent and portioned, the organ (such as a metanephros) attached to the animal body (such as a fetus body) is punctured with the needle-shaped member to inject the foreign substance (for example, see Non-Patent Document 2). Further, in a case that the organ is a metanephros, a position of a nephrogenic zone can be easily specified in a state in which the metanephros is attached to the animal body (such as a fetus body), which is also a reason to adopt the method.

### CITATION LIST

### Patent Document

Non-Patent Document 1: Yokoo Takashi et al., “Journal of Clinical and Experimental Medicine (IGAKU NO AYUMI)”, Volume 279, Issue 7, 715-719 (2021)
Non-Patent Document 2: M Yamanaka et al., Nature Communications, 2017: p. 12, Injection of NPCs into nephrogenic zone

### DISCLOSURE OF THE INVENTION

### Problems to be Solved by the Invention

However, the above method has the following problems.
1) Since an injecting operation can be performed only in the state where an organ such as a metanephros is attached to the animal body such as a fetus body, there are large restrictions on a place and a time for the injecting operation.
2) Since the organ such as a metanephros removed from the animal body such as a fetus body is an organ that is very small (about a major axis of 2 mm×a minor axis of 1 mm), it is difficult to puncture the needle-shaped member while holding the organ softly so as not to damage the organ.
3) When an injection target zone in the organ such as a metanephros is present on a side on which the injection target zone is not exposed after the animal body (such as a fetus body) is incised, it cannot be avoided to puncture the needle-shaped member such as a capillary tube from an exposed surface side of the organ and penetrate to the injection target zone to inject the foreign substance. Accordingly, a transplant material is seriously damaged by an injection path.

The present invention has been made in view of the above problems in the related art, and a first object thereof is to provide a method for producing a transplant material that allows easy injection of a foreign substance into an organ removed from an animal body. Further, a second object of the present invention is to provide a transplant material to which damage is reduced even when the foreign substance is injected.

### Means for Solving the Problems

As a result of intensive studies on the above problems, the present inventors have discovered that by bringing at least a portion of an organ removed from an animal body into contact with a member having plasticity and positioning the organ, it is possible to easily inject a foreign substance to a desired portion of the organ through a shortest path without requiring a complicated operation. The present invention has been completed based on such discovery. That is, the present invention is as follows.
<1> A method for producing a transplant material including an organ into which a foreign substance is injected includes: a step of bringing at least a portion of an organ removed from an animal body into contact with a member having plasticity and positioning the organ; and a step of injecting a foreign substance into the positioned organ.
<2> The method described in <1>, in which the injection is performed from a surface immediately above or near an injection target zone in the organ.
<3> The method described in <1> or <2>, in which the animal body is a fetus body.
<4> The method described in any one of <1> to <3>, in which the organ is an organ for xenotransplantation.
<5> The method described in any one of <1> to <4>, in which the organ is an organ subjected to vitrification freezing after being removed.
<6> The method described in <5>, in which the member having plasticity is a member having plasticity covering the organ during the vitrification freezing.
<7> The method described in any one of <1> to <6>, in which at least a portion of the organ is embedded in the member having plasticity.
<8> The method described in any one of <1> to <7>, in which the member having plasticity is a gel.
<9> The method described in any one of <1> to <8>, in which a needle-shaped member is used for the injection.
<10> The method described in <9>, in which a path through which the needle-shaped member passes has a dense structure that is difficult for a liquid to penetrate.
<11> The method described in any one of <1> to <9>, in which the foreign substance refers to a cell derived from an animal other than an animal of the animal body.
<12> The method described in any one of <1> to <10>, in which the organ is a kidney, and the injection target zone is a nephrogenic zone of a metanephros.
<13> A transplant material includes: an organ removed from an animal; and a foreign substance injected into the organ, an injection mark is formed on the organ immediately above or near a zone in which the foreign substance is injected.
<14>The material described in <13>, in which the foreign substance refers to a cell derived from a portion other than the organ.

### Effects of the Invention

According to the present invention, it is possible to provide a method for producing a transplant material that allows easy injection of a foreign substance into an organ removed from an animal body. Further, according to the present invention, it is possible to provide a transplant material to which damage is reduced even when the foreign substance is injected. Further, according to the present invention, it is possible to reduce restrictions on a place and a time for an injecting operation.

### BRIEF DESCRIPTION OF THE DRAWINGS

FIG. 1 is a schematic view illustrating a preferable embodiment of the present invention,

FIG. 2 is a diagram illustrating a microscope photograph of a metanephros that is positioned by semi-embedding into a gelatin gel,

FIG. 3 is a diagram illustrating a microscope photograph that shows a result of injection of a foreign substance (GFP-expressing cells) into one location in a nephrogenic zone under a renal capsule,

FIG. 4 is a diagram illustrating a microscope photograph that shows a result of injection of the foreign substance (GFP-expressing cells) into two location in the nephrogenic zone under the renal capsule,

FIG. 5 is a diagram illustrating a microscope photograph that shows a result of injection of the foreign substance (GFP-expressing cells) into a metanephros mesenchyme zone in a wide range,

FIG. 6 is a diagram illustrating a microscope photograph that shows an injection result of Example 3,

FIG. 7A and FIG. 7B are diagrams illustrating a microscope photograph that shows an injection result of Comparative Example 1,

FIG. 8 is a diagram illustrating pancreas tissues of a pig in which B cells were lacking due to

expression of a Pdx1-Hes1 gene in Example 4; and

FIGS. 9A-FIG. 93 are diagrams illustrating a state of a human iPS cell-derived pancreatic progenitor cell mass injected into one of a plurality of pancreas tissue fragments illustrated in FIG. 8.

### PREFERRED MODE FOR CARRYING OUT THE INVENTION

Hereinafter, an embodiment of the present invention will be described in detail, but the present invention is not in any way limited to the following embodiment and can be implemented with appropriate modifications within the scope of the object of the present invention.

### <<Method for Producing Transplant Material Including Organ Into Which Foreign Substance is Injected>>

A first aspect of the present invention relates to a method for producing a transplant material including an organ into which a foreign substance is injected that includes: a step of bringing at least a portion of an organ removed from an animal body into contact with a member having plasticity and positioning the organ; and

a step of injecting a foreign substance into the positioned organ. According to the first aspect, by bringing at least a portion of the organ removed from the animal body into contact with the member having plasticity and positioning the organ, it is possible to easily inject the foreign substance into the organ removed from the animal body without requiring a complicated operation.

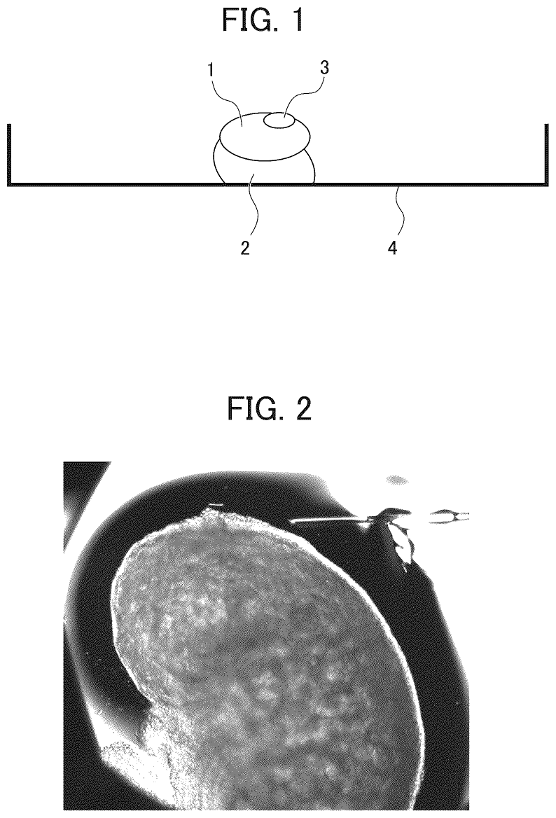

A preferable embodiment will be described with reference to FIG. 1. By bringing (preferably, embedding) at least a portion of an organ 1 removed from the animal body into contact with (into) a member having plasticity 2 and positioning the organ 1, it is possible to easily (preferably, precisely) inject the foreign substance into an injection target zone 3 of the organ 1 by any means. The member having plasticity 2 is preferably brought into contact with (for example, provided on, fixed to) a substrate 4.

Examples of animals that provide the animal body include any mammals such as pigs, cattle, horses, sheep, goats, primates (for example, humans and anthropoid apes (such as chimpanzees and monkeys), and rodents (such as mice and rats). As the animals, the mammals having features such as a physique closer to humans than the rodents are preferable, pigs, sheep, goats, and the primates (for example, humans and anthropoid apes) are more preferable, and pigs (that is, the organ is an organ obtained from a pig) are further preferable. As the animal body, although any one of a fetus body, a young animal body, and an adult animal body may be used, the animal body is preferably a fetus body from the viewpoint of low immunogenicity. In particular, when the organ is an organ for xenotransplantation, the animal body is preferably a fetus body.

The organ is preferably an organ for transplantation and is more preferably an organ for xenotransplantation. Specific examples of the organ include viscera (for example, pancreas, kidney, ureter, bladder, liver, heart, stomach, and intestine), genitals (for example, ovary and testicle), zygotes, embryos, fetuses, bone marrows (for example, hematopoiesis), brains, eyes, noses, mouths, skins, nerves, and tissues thereof or artificial tissues (cell sheets such as a chondrocyte sheet, organoids and the like). As the organ, the viscera, the genitals, the zygotes, the embryos, and the fetuses are preferable, the viscera and the genitals are more preferable, the pancreas (for example, a pancreatic islet), the kidney (for example, a metanephros, particularly, urinary organs including a metanephros, a ureter and a bladder) are further preferable.

The organ may or may not be a genetically modified organ (a recombinant organ or a genome-edited organ). Examples of preparation of the genetically modified organ include (1) preparation of genetically modified animal cells and (2) preparation of the organ based on the prepared cells according to somatic cell clone technique. Examples of (1) the preparation of the genetically modified animal cells include a method for injecting a vector containing a desired transformer gene or a genome editing tool into an animal zygote, and an ICSI mediated gene transfer method based on microinsemination (intra-cytoplasmic sperm injection; also referred to as ICSI) between a liquid containing animal sperms and an egg cell (preferably an ovum, and more preferably a mature ovum). Further, the preparation of the cells may or may not be introduction of a desired transformer gene according to a gene editing technique such as clusterd regularly interspaced short palindromic repeats (CRISPR)/Cas nuclease.

Regarding (2) the preparation of the genetically modified organ according to the somatic cell clone technique based on the genetically modified cells prepared, the somatic cell clone technique refers to a technique of culturing a cell of an animal for which a clone is desired to be prepared (preferably the genetically modified animal cell) as a donor cell, performing nuclear transplantation on the donor cell by, for example, a fusion method such as cell fusion in an enucleated recipient egg (an oocyte) of a recipient, and culturing the cell, and then implanting the cell in a foster mother and enabling conception to produce a clone. According to the somatic cell clone technique, it is possible to clone a genetically modified animal and prepare a genetically modified organ by removing a target organ from the animal.

The organ may or may not be a secondary organ. Here, the “secondary organ” refers to an organ-like tissue formed by culturing and growing an animal at an in-vivo site at which an organ (for example, the pancreas) whose formation is hindered is originally intended to be formed (see JP 2019-62929A). The secondary organ may or may not be the genetically modified organ. That is, the hindering of the organ formation can be performed by genetic modification for modifying a gene of an animal having organs. For example, when the formation of the pancreas is hindered, the hindering can be performed by using a Pdx1-Hes1 gene in which an Hes1 gene is collected to a promoter of a Pdx1 gene, that is, by overexpressing the Hes1 gene under the control of a Pdx1 promoter (see Matsunali et al., PNAS 110:4557-4562 (2013); JP 2019-62929A). Further, the secondary organ may be formed by hindering the organ formation via drug administration.

In a case of hindering formation of the kidney, the hindering can be performed by genetic modification for overexpressing a Six2-Notch2 gene (Fujimura et al., J Am Soc Nephrol, 21: 803-810, 2010) or by hindering or restraining expression of genes such as Sall 1 and Pax2 that control kidney development. The secondary organ may have 100% of a function of an original organ or may have only a part thereof. Preferable examples of the secondary organ can include a secondary organ lacking a specific functional cell. Here, the “functional cell” refers to a cell that has some function among cells contained in the organ. For example, examples thereof include α cells, β cells, and γ cells in the pancreas, and examples thereof include nephron progenitor cells, metanephros mesenchymal cells, and ureteric bud cells in the kidney. Preferable examples of the functional cell include the β cells in the pancreas, and the nephron progenitor cells in the kidney. Preferable examples of the organ lacking a specific functional cell include a pancreas lacking β cells, a kidney lacking nephron progenitor cells, and a kidney lacking metanephros mesenchymal cells.

When the organ is the kidney (for example, a metanephros, particularly, urinary organs including a metanephros, a ureter, and a bladder), a kidney after a metanephros (kidney primordia) is formed at a tip of a ureter is preferable, and a kidney (for example, a metanephros, particularly, urinary organs including a metanephros, a ureter, and a bladder) corresponding to an extension stage of a ureteric bud that serves as a main element for forming nephron later is more preferable. More specifically, a kidney (for example, a metanephros, particularly, urinary organs including a metanephros, a ureter, and a bladder) having a fetal age of 25 days to 45 days corresponding to the extension stage is preferable, and a kidney (for example, a metanephros, particularly, urinary organs including a metanephros, a ureter, and a bladder) having a fetal age of 30 days to 40 days is more preferable.

Examples of the aspect of bringing at least a portion of the organ into contact with the member having plasticity include: placing the organ on the member having plasticity, and firmly adhering at least a portion of the organ to a lower-side portion, a lateral-side portion, or an upper-side portion of the adhesive member having plasticity by embedding or the like so as not to slip therefrom. In any one of the aspects, at least a portion of the organ is preferably embedded in the member having plasticity. The portion of the organ may be partially embedded in the member having plasticity and be partially exposed, and the organ may be completely embedded in the member having plasticity (may be completely covered with the member having plasticity). Further, regarding the aspect of bringing at least a portion of the organ into contact with the member having plasticity, when the member having plasticity is a gel to be described later, the aspect may or may not refer to performing gelation after immersing the organ into a liquid (preferably a hydrosol) containing a sol (a sol of the gel before the gelation), performing the gelation after coating the liquid containing the sol to the organ, or the like. The gelation of the sol can be performed by any method such as addition and cooling of polyvalent metal ions such as calcium ions and barium ions. Here, the “positioning” refers to restraining movement of the organ due to slipping or the like and maintaining a positional relation of the organ with respect to the member having plasticity.

The member having plasticity is not particularly limited as long as the member having plasticity has plasticity (deformability and flexibility), which allows adherence to the organ to such an extent that the movement of the organ due to the slipping or the like can be restrained, and can achieve the objects of the present invention, and any member containing a plastic material can be included. A member containing a plastic material having a friction coefficient, which allows restraint of the movement of the organ, is preferable, and for example, a member containing a plastic adhesive material, or the like may or may not be used. From the viewpoint that no actual damage occurs even when the plastic material is not completely removed and a small amount of the plastic material remains, and that the plastic material can be used for transplantation in a coated state without being removed, a member containing any pharmacologically acceptable plastic material (pharmacologically acceptable non-toxic plastic material) is preferable. Particularly, from the viewpoint of having the plasticity (the deformability and the flexibility), which allows adherence to the organ to such an extent that the movement of the organ can be restrained, and having an appropriate friction coefficient due to a hydrogen bond, an intermolecular force, or the like, a gel is preferable, any pharmacologically acceptable gel (pharmacologically acceptable non-toxic gel) is more preferable, and a hydrogel is still preferable.

Further, from the viewpoint of easy control of sol-gel phase transition, the gel is preferably a gel in which the sol-gel phase transition occurs due to ion-crosslinking, or a temperature-sensitive gel in which the sol-gel phase transition occurs due to a temperature (for example, a phase transition temperature of 5° C. to 40° C., preferably a phase transition temperature of 5° C. to 10° C., 10° C. to 15°0 C., 15° C. to 20° C., 20° C. to 25° C., 25° C. to 30° C., 30° C. to 35° C., or 35° C. to 40° C.). The gel in which the sol-gel phase transition occurs due to the ion-crosslinking can decompose the ion-crosslinking and be subjected to the solation by a chelation treatment based on a chelating agent such as ethylenediamine tetraacetic acid (EDTA), ethylenediamine tetraacetic acid disodium (EDTA·2Na).

Examples of the hydrogel include alginic acid and salts thereof (For example, polyvalent metal salts such as a calcium alginate and a barium alginate, and monovalent metal salts such as a sodium alginate and a potassium alginate are included. From the viewpoint of more reliably achieving the gelation, the polyvalent metal salts such as a calcium alginate and a barium alginate are preferable.), gelatin, carrageenan, agar (agarose gel), pectin, chitosan, silicone hydrogel, konjac, and other polysaccharides, among these, a hydrogel in which the sol-gel phase transition occurs due to the ion-crosslinking or a temperature-sensitive hydrogel in which the sol-gel phase transition occurs due to a temperature is preferable. Examples of the hydrogel in which the sol-gel phase transition occurs due to the ion-crosslinking include alginic acid and salts thereof (preferably polyvalent metal salts). Examples of the temperature-sensitive hydrogel in which the sol-gel phase transition occurs due to a temperature include gelatin, carrageenan, agar (agarose gel), and pectin. The gelatin is preferable because the gelatin can be subjected to the solation at a relatively low temperature (for example, 35° C. or lower) and causes less damage to the organ or the like. For example, a 5% (w/v) to 25% (w/v) gelatin solution can be used. A 7% (w/v) to 20% (w/v) gelatin solution is preferable, and a 10% (w/v) to 15% (w/v) gelatin solution is more preferable. A size of the member having plasticity can be appropriately set by a person skilled in the art according to a size of the organ, for example, when a cross section of the organ is regarded as an ellipse, the member having plasticity can be set to a sheet-like member having a long side and a short side 0.25 times to 3 times a long axis and a short axis of the organ, respectively. A sheet-like member having a long side and a short side 0.35 times to 2 times the long axis and the short axis of the organ respectively is preferable, and a sheet-like member having a long side and a short side 0.5 times to 1.5 times the long axis and the short axis of the organ respectively is more preferable. More specifically, a sheet-like member having a long side of 10 mm to 200 mm and a short side of 5 mm to 100 mm is included. A thickness thereof can be set to, for example, 0.1 mm to 5 mm (preferably 0.5 mm to 2 mm).

A method for injecting the foreign substance is not particularly limited as long as the foreign substance can be injected into the organ, and examples thereof include a method for puncturing a surface of the organ with a needle-shaped member to form a hole, and then injecting the foreign substance from the hole by any means. A method for puncturing the surface of the organ with a hollow needle-shaped member and then injecting the foreign substance by any means (for example, capillary phenomenon, injection) is preferable. A hole diameter (for example, an average diameter) of the hole is not particularly limited, examples thereof include 0.05 mm to 3 mm, 0.1 mm to 2 mm is preferable, and 0.5 mm to 1.5 mm is more preferable. Examples of the needle-shaped member include a capillary tube having a sharp tip. An operation of injecting the foreign substance may or may not be performed by a freehand (for example, a freehand under a microscope by a skilled operator), and from the viewpoint of more accurately executing the operation, the operation is preferably executed by using a micromanipulator. Here, the foreign substance refers to a substance derived from a portion other than the organ, examples of the foreign substance include a substance derived from a portion that is the same animal species as a recipient (a recipient individual), a substance derived from a portion that is the same animal species as a donor (a provider individual) but is an individual different from the donor, a substance derived in a case in which the recipient and the donor are different animal species, and drugs (for example, medicines). As a substance derived from an animal species, a substance derived from a portion that is the same animal species as a recipient is preferable from the viewpoint of low immunogenicity. Examples of the substance include cells, growth factors, hormones, and cytokines, and among these, the cells are preferable. The cells may or may not be artificial cells (for example, gene recombinant cells, ES cells, and iPS cells). For example, in a case of xenotransplantation for transplanting a pig organ into a human, examples of the “cell derived from a portion that is the same animal species as a recipient” include “cells derived from a human”.

The injection is preferably performed from a surface immediately above or near an injection target zone in the organ. Here, the “surface near the injection target zone” refers to a surface of the organ that presents on a side same as the injection target zone with respect to a center of the organ. A surface of the organ including a point on an extension line connecting the center and any one point in the injection target zone is preferable. Further, the “surface immediately above the injection target zone” refers to a surface closest to the injection target zone on the surface of the organ. The “surface near the injection target zone” is preferably a zone of a surface including the “surface immediately above the injection target zone”. Examples of the injection target zone include any zone in the organ. For example, when the organ is a kidney, examples of the injection target zone include a metanephros nephrogenic zone and a metanephros mesenchyme zone. The substrate 4 is not particularly limited, and examples thereof include a plate (made of plastic, metal, or the like), a well, a glass substrate, and a tray (for example, a dish). At least a portion of an injection mark formed on the organ may or may not be covered with the member having plasticity. The “injection mark” will be described in detail later.

The organ may or may not be an organ subjected to vitrification freezing after being removed. According to the present invention, a place and/or a time for each of the removal of the organ from the animal body and the injection of the foreign substance can be independent, and thus an organ conveyed and/or preserved in a state of maintaining quality by the vitrification freezing can be preferably used. Examples of the organ subjected to the vitrification freezing include an organ subjected to vitrification cryopreservation (for example, preserved for a long period of time). The organ may be covered with the member having plasticity during the vitrification freezing. In this case, the member having plasticity covering the organ during the vitrification freezing may be the same as or different from the member having plasticity (a member functioning as a positioning tool) in the first aspect. From the viewpoint of step simplification, it is preferable to use, as the member having plasticity (the member functioning as a positioning tool) in the first aspect, the member having plasticity covering the organ during the vitrification freezing as it is.

As described above, in the related art, in a state in which the animal body (such as a fetus body) is incised to some extent and portioned, the organ (such as a metanephros) attached to the animal body (such as the fetus body) is punctured with the needle-shaped member to inject the foreign substance (for example, see Non-Patent Document 2). As a result, according to the above known method, it is necessary to penetrate a kidney parenchyma from a direction of a renal hilus having a large number of voids such as a renal pelvis and having important vessels such as a renal artery, a renal veins, and a ureter (puncture the kidney parenchyma with the needle-shaped member) to inject the foreign substance. As a result, since the renal hilus has a large number of the vessels and the voids, there is a problem that the foreign substance after the injection leaks out. In addition, the renal hilus is an important tissue at which the important vessels such as a renal artery, a renal veins, and a ureter are aggregated with the kidney development, and thus there is also a problem that although it is necessary to avoid damage to a renal hilus portion as much as possible at the time of injecting the foreign substance into the metanephros, the known method for injecting the foreign substance from the direction of the renal hilus has high invasiveness to a renal tissue.

In the present invention, as described above, at least a portion of the injection mark formed on the organ may or may not be covered with the member having plasticity. According to the present invention, it is possible to avoid the injection of the foreign substance from a direction in which a large number of the vessels and the voids are present such as the direction of the renal hilus even when the portion is not covered with the member having plasticity as described above, and thus the leakage of the foreign substance during the vitrification freezing can be restrained regardless of whether the injection mark is covered. Further, according to the present invention, the damage to the renal hilus portion can be avoided, and thus the invasiveness is lower than that of the known method. In the present invention, in particular, in a case in which the injection is performed by the needle-shaped member, when a path through which the needle-shaped member passes inside the organ (a path in which a communication hole to be described later is formed) has a dense structure, the leakage of the foreign substance can be restrained. For example, it is for a reason that the path is filled back to a certain extent by a tissue or cells. Examples of the above dense structure include a structure (for example, a structure of a parenchyma portion of an organ (a parenchymal organ, a solid organ, and the like)) in which the number of vessels, voids, or the like is less than that of a luminal portion of an organ (for example, a luminal organ), a hilus portion, or the like, or in which no vessel, void, or the like is present. A dense structure is preferable that is difficult for a liquid (preferably, a liquid containing the foreign substance) to penetrate as compared with the luminal portion of the organ (for example, the luminal organ), the hilus portion, or the like, and a structure in which a tissue (for example, a connective tissue) or cells (for example, mesenchymal cells) are densely filled is more preferable. When the organ is a metanephros, it is preferable that the organ has the metanephros mesenchyme zone, the nephrogenic zone, and a structure in which metanephros mesenchymal cells such as Cap mesenchyme are filled.

Here, the vitrification freezing refers to bringing the organ into contact with a vitrification freezing refrigerant such as liquid nitrogen (a boiling point of—196° C.), liquid helium (a boiling point of—269° C.), and liquid ethane (a boiling point of—175° C.) to restrain crystallization of water (formation of ice crystals), and freezing (preferably, freezing by so-called rapid cooling, more preferably, freezing by so-called ultra-rapid cooling) the organ in an amorphous glass state. According to the vitrification freezing, volume expansion associated with the crystallization of water can be restrained, and thus damage (freezing damage) such as breakage of a cell membrane can be restrained. A method for the contact with the vitrification freezing refrigerant is not particularly limited, and examples thereof include immersion or application (flash) of a liquid and blowing of steam. The contact with the vitrification freezing refrigerant may or may not be performed by using a vitrification freezing support tool capable of supporting a plurality of the organs simultaneously or a vitrification freezing support tool such as a Cryotop, a straw (for example, a transplant straw and a straw having a sharp tip), and a capillary pipette, and the contact with the vitrification freezing refrigerant is preferably performed by using the support tool. Regarding the support tool, the vitrification freezing support tool capable of supporting the plurality of organs simultaneously is more preferable from the viewpoint that the plurality of organs are integrally frozen at one time to improve production efficiency of the transplant material. For example, examples of the vitrification freezing support tool capable of supporting the plurality of organs simultaneously include a chip or a plate having a plurality of recesses on a glass plate, a metal plate, a plastic plate, or the like and capable of simultaneously supporting the plurality of organs in the recesses, and

a support tool capable of simultaneously supporting the plurality of organs on a mesh-like net, a nonwoven fabric, or the like. Further, examples of a commercially available support tool include CRYOTOP (registered trademark: manufactured by KIRAZATO BioPharma Co., Ltd.).

The organ is preferably brought into contact (immersed, coated, or the like) with any vitrification solution before the vitrification freezing. The vitrification solution is not particularly limited, and examples thereof include a solution (preferably an aqueous solution) containing a cell-permeable cryoprotective agent such as dimethyl sulfoxide (DMSO), ethylene glycol (EG), propanediol, and glycerin: a saccharide such as sucrose, trehalose, sorbitol, and dextran: and a cell-impermeable cryoprotective agent such as carboxylated polylysine, polyvinyl alcohol, polyvinyl pyrrolidone, and antifreeze proteins. A pretreatment for bringing (immersing, coating, or the like) the organ into contact with any equilibrium solution may or may not be performed before bringing the organ into contact with the vitrification solution.

After the above freezing step, the organ subjected to the vitrification freezing (preferably, vitrification cryopreservation) may or may not be brought into contact (immersed, coated, or the like) with any thawing solution. After being brought into contact with the thawing solution, the organ may or may not be brought into contact (immersed, coated, or the like) with any diluent.

In the preferable embodiment, it is preferable to further include a step of bringing the organ into contact with the vitrification solution after a covering step from the viewpoint of restraining the leakage of the foreign substance due to an osmotic pressure gradient.

The organ subjected to the above treatment may or may not be cultured in a basal medium or a basal medium liquid before transplantation (preferably xenotransplantation).

For example, in a case in which the organ is used as a product for regenerative medicine, it may be required to inject the foreign substance (for example, the cells derived from a portion that is the same animal species as a recipient) into the organ and then perform the vitrification freezing on the organ and preserve the organ. On the other hand, when the injection mark to be described later is present, there is a problem in the related art that the foreign substance leaks out from the organ due to an osmotic pressure gradient between the inside of the organ and an external solution (for example, the vitrification-process solution, the equilibrium solution, and the like) during the vitrification-freezing process. Further, the vitrification freezing refrigerant and the vitrification-process solution are repeatedly used or are simultaneously used for a plurality of samples, which may lead to a risk of contamination (for example, cell contamination). Therefore, the organ may or may not be covered with the member having plasticity during the vitrification freezing from the viewpoint of reducing the leakage of the foreign substance and reducing the risk of contamination. In order to cover the organ during the vitrification freezing, the member having plasticity (the member used as the positioning tool) according to the first aspect may be used as it is. According to the present invention, it is possible to avoid the injection of the foreign substance from the direction in which a large number of the vessels and the voids are present such as the direction of the renal hilus regardless of whether the injection mark is covered, and thus the leakage of the foreign substance during the vitrification freezing can be restrained. In the present invention, in particular, in the case in which the injection is performed by the needle-shaped member, when the path through which the needle-shaped member passes inside the organ (the path in which the communication hole to be described later is formed) has a dense structure that is difficult for a liquid to penetrate, the leakage of the foreign substance can be restrained.

In view of the above problem, it is also a preferable embodiment that the organ has a foreign substance injected therein and an injection mark, and the member having plasticity covers at least a portion of the injection mark. Here, the term “injection mark” refers to a hole formed for artificially injecting the foreign substance into the organ and refers to not only the hole on the surface of the organ but also the communication hole that extends up to the injection target zone inside the organ. The “injection mark” may be formed by, for example, the needle-shaped member or the like. According to the preferable embodiment, at least a part of the injection mark including the hole on the surface and the communication hole, and the hole on the surface may be covered with the gel. As a result, it is possible to restrain the leakage of the foreign substance due to the osmotic pressure gradient between the inside of the organ and the external solution (for example, the vitrification solution, the equilibrium solution, and the like).

After the freezing step, the method may or may not include a removing step of removing the member having plasticity covering the surface of the organ. From the viewpoint that the organ after the member having plasticity is removed can be used for transplantation as a transplant donor, it is preferable to include the removing step. When the covered member having plasticity is a gel, the gel can be removed by heating the member having plasticity to a solation temperature, by performing any solation such as the chelation treatment, by a physical pressure, or the like. Further, the organ may or may not be washed by using any aqueous solution or any cleaning liquid such as a phosphate buffer solution, (running water). The removing step may or may not be performed multiple times (for example, stepwise).

### <<Transplant Material>>

The second aspect of the present invention is a transplant material that includes: an organ removed from an animal; and a foreign substance injected into the organ, an injection mark is formed on the organ immediately above or near a zone in which the foreign substance is injected. Specific examples and preferable examples of the organ are as described above. It is preferable that the organ has the foreign substance injected therein and the injection mark, and the gel covers at least a portion of the injection mark, it is more preferable that the gel covers the entire injection mark, and it is still preferable that the gel covers the entire surface of the organ. Specific examples and preferable examples of the foreign substance are as described above. The injection mark is described as above.

### EXAMPLES

Examples of the present invention will be described below to further specifically explain the present invention, but the present invention is not limited to these examples, and various applications can be made without departing from the technical ideas of the present invention.

### (Materials)

A capillary tube having a sharply polished tip was prepared, and the capillary tube sucked GFP expression-human mesenchymal stem cells that express green fluorescent proteins (GFP) and emit green fluorescence. On the other hand, a gelatin powder (manufactured by FUJIFILM Wako Pure Chemical Corporation) was dissolved in a HEPES-buffered TCM199 medium at pH 7.2 to prepare a gelatin solution having a concentration of 10% (w/v) to 15% (w/v).

### Example 1

A metanephros was removed from a pig fetus obtained from a DPF-controlled wild type mother pig by caesarean section, the metanephros was placed in the gelatin solution, and the metanephros was semi-embedded (that is, a portion of the metanephros was not in contact with the gel, and a portion of the metanephros was in contact with the gel was embedded) in the gelatin gel such that the surfaces immediately above and near the nephrogenic zone were exposed.

### Positioning Step

FIG. 2 is a diagram illustrating a microscope photograph of the metanephros that is positioned by semi-embedding into the gelatin gel. As illustrated in FIG. 2, the metanephros semi-embedded in the gelatin gel was placed on a 35 mm dish (manufactured by Iwaki Co., Ltd.) and is positioned such that the surfaces immediately above and near the nephrogenic zone were exposed (faced an upper side opposite to the dish).

### Foreign Substance Injecting Step

The capillary tube sucking the GFP expression-human mesenchymal stem cells was punctured to one or two locations on the surface immediately above or near the nephrogenic zone under a capsule of the semi-embedded metanephros such that a distance to the nephrogenic zone was shortest, and the human mesenchymal stem cells as the foreign substance were injected into the nephrogenic zone. Further, after being positioned such that surfaces immediately above and near a mesenchyme zone of the semi-embedded metanephros were exposed (faced the upper side opposite to the dish), the capillary tube was punctured in a wider range such that a distance to the mesenchyme zone was shortest, and the tip of the capillary tube was moved to inject the human mesenchymal stem cells into the mesenchyme zone in a wider range.

### Results

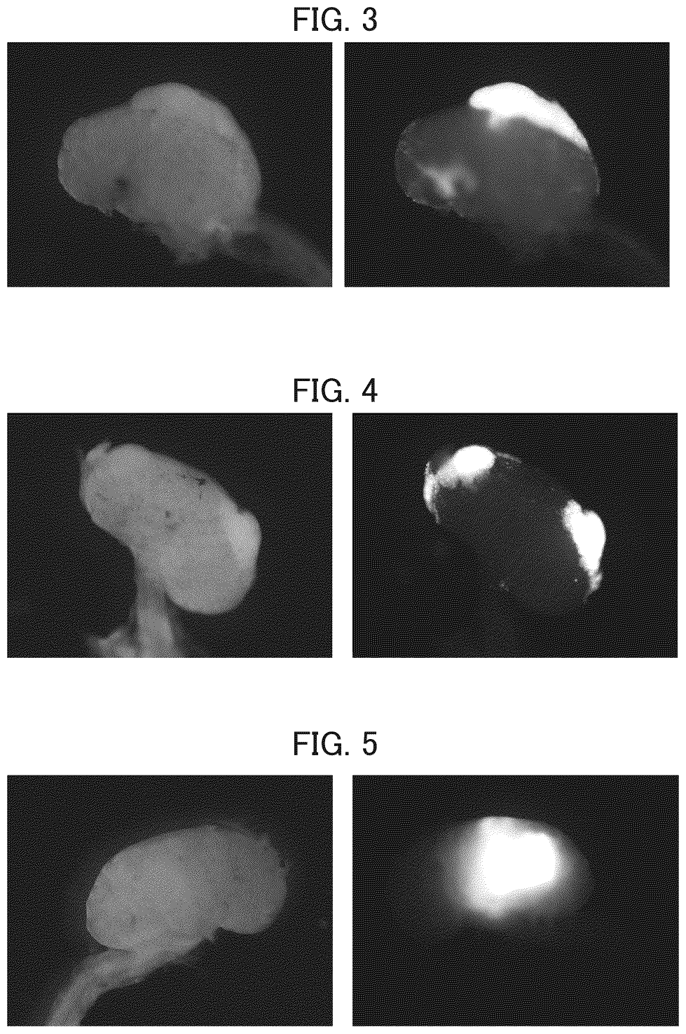

Results are shown in FIGS. 3 to FIG. 5. FIG. 3 is a diagram illustrating a microscope photograph that shows a result of injection of GFP-expressing cells into one location in the nephrogenic zone under the capsule of the semi-embedded metanephros. FIG. 4 is a diagram illustrating a microscope photograph that shows a result of injection of the GFP-expressing cells into two locations in the nephrogenic zone under the capsule of the semi-embedded metanephros. FIG. 5 is a diagram illustrating a microscope photograph that shows a result of injection of the GFP-expressing cells into the metanephros mesenchyme zone in a wide range. As is clear from the results shown in FIGS. 3 to FIG. 5, it can be understood that the foreign substance (the GFP-expressing cells) could be easily injected into a desired injection target zone of the organ removed from the animal body. Further, it can be said that it is possible to provide a transplant material in which the foreign substance is injected and the damage is reduced by injecting the foreign substance at the shortest distance to the surfaces immediately above and near the injection target zone.

### Example 2

A metanephros was removed from a pig fetus obtained from a DPF-controlled wild type mother pig by caesarean section, the metanephros was placed in the gelatin solution, and the entire metanephros was covered with the gelatin gel. The metanephros completely covered with the gelatin gel was immersed in 4.5 ml of an equilibrium solution containing 7.5 mass % of EG and 7.5 mass % of DMSO as cryoprotective agents for 25 minutes at room temperature (25° C. to 27° C.) and was subjected to the pretreatment. Then, the metanephros after the pretreatment was immersed in a vitrification solution containing 15 mass % of EG, 15 mass % of DMSO, which are double the above amounts, and 0.5 M of sucrose for 30 minutes at the room temperature. Further, the metanephros after being immersed in the vitrification solution was held on a support tool “Cryotop” (manufactured by KIRAZATO BioPharma Co., Ltd.) and was placed in liquid nitrogen to be ultra-rapidly cooled and subjected to the vitrification freezing. The metanephros after the vitrification freezing was transferred into a vitrification cryopreservation container and cryopreserved in the liquid nitrogen for 10 days, and then was thawed by being immersed in a 37° C. thawing solution containing 1 M of sucrose for 1 minute. Then, the metanephros was washed twice with a diluent containing 0.5 M of sucrose for 3 minutes at the room temperature and twice with a cleaning liquid for 5 minutes to dilute and remove the cryoprotective agents.

### Positioning Step

The metanephros subjected to the vitrification freezing and the thawing and completely covered with the gelatin gel was placed on a 35 mm dish (manufactured by Iwaki Co., Ltd.), and was positioned such that the surfaces immediately above and near the nephrogenic zone faced an upper side opposite to the dish.

### Foreign Substance Injecting Step

The capillary tube sucking the GFP expression-human mesenchymal stem cells was punctured to one or two locations on the surface immediately above or near the nephrogenic zone under a capsule of the completely covered metanephros such that a distance to the nephrogenic zone was shortest, and the human mesenchymal stem cells as the foreign substance were injected into the nephrogenic zone. Further, after being positioned such that surfaces immediately above and near a mesenchyme zone of the completely covered metanephros faced an upper side opposite to the dish, the capillary tube was punctured in a wider range such that a distance to the mesenchyme zone was shortest, and the tip of the capillary tube was moved to inject the human mesenchymal stem cells into the mesenchyme zone in a wider range.

### Results

As in Example 1, a microscope photograph that shows the result of the injection of the GFP-expressing cells into one or two locations in the nephrogenic zone under the capsule of the metanephros was obtained. Further, as in Example 1, a microscope photograph that shows the result of the injection of the GFP-expressing cells into the metanephros mesenchyme zone in a wide range was obtained. It can be understood from the results that the foreign substance (the GFP-expressing cells) could be easily injected into a desired injection target zone of the organ removed from the animal body. Further, it can be said that it is possible to provide a transplant material in which the foreign substance is injected, and the damage is reduced by injecting the foreign substance at the shortest distance to the surfaces immediately above and near the injection target zone.

### Example 3 and Comparative Example 1

### Example 3

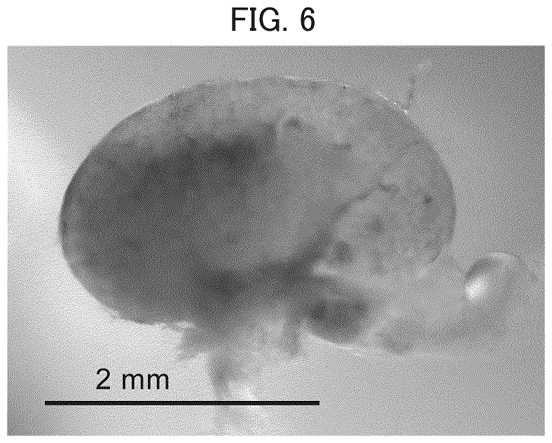

As in Example 1, a metanephros was removed from a pig fetus obtained from a DPF-controlled wild type mother pig by caesarean section and having an age of 35 days, the metanephros was placed in the gelatin solution, and the metanephros was semi-embedded (that is, a portion of the metanephros was not in contact with the gel, and a portion of the metanephros was in contact with the gel was embedded) in the gelatin gel such that the surfaces immediately above and near the nephrogenic zone were exposed. As in Example 1, the metanephros semi-embedded in the gelatin gel was placed on a 35 mm dish (manufactured by Iwaki Co., Ltd.) and was positioned such that the surfaces immediately above and near the nephrogenic zone were exposed (faced an upper side opposite to the dish). A capillary tube sucking 0.1 μL of an edible green dye solution was punctured to one location on the surface immediately above or near the nephrogenic zone under a capsule of the semi-embedded metanephros such that a distance to the nephrogenic zone was shortest, and 0.1 μL of the edible green dye solution as the foreign substance was injected into the nephrogenic zone. The metanephros having an injection mark after the dye injection (the injection mark was not covered with the gel) was left standing in a physiological saline, and then leakage of the dye was observed under a stereomicroscope at 10 minutes after the injection. A result thereof was shown in FIG. 6 (the number of n=3).

### Comparative Example 1

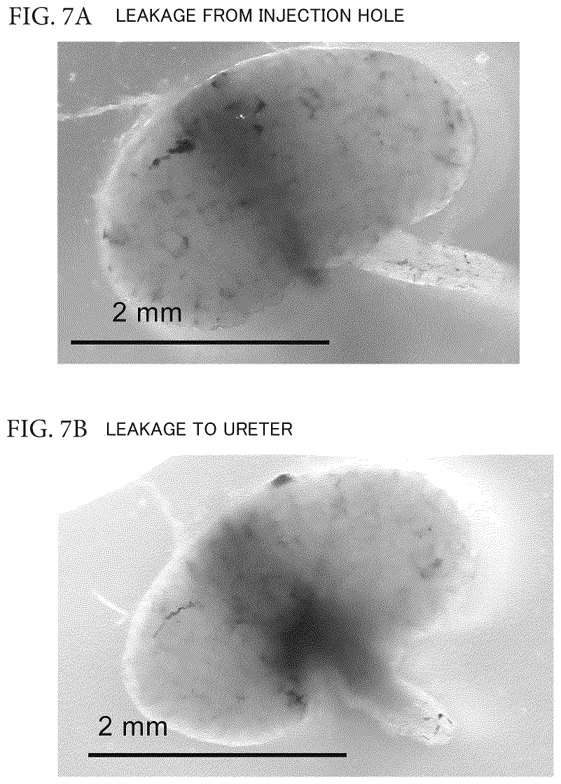

A pig fetus obtained from a DPF-controlled wild type mother pig by caesarean section and having an age of 35 days was killed by decapitation. As described in Non-Patent Document 2, joint portions of both hindlimbs were incised from a caudal side along a spinal cord. Similarly, another side surface (a back surface) was also cut open and vertebrae were taken out from the body. After a pair of kidneys (metanephroi) were visible inside the body, the fetus was fixed with microtweezers. Thereafter, the capillary tube sucking 0.1 μL of the edible green dye solution was punctured from a renal hilus toward a direction of the renal capsule and was carefully injected so as not to rupture the renal capsule. The kidneys (metanephroi) after the injection were separated from the fetus by using the microtweezers in a state in which a ureter and a bladder were still connected. The metanephroi each having an injection mark after the dye injection (the injection mark was not covered with the gel) were left standing in a physiological saline, and then leakage of the dye was observed under a stereomicroscope at 10 minutes after the injection. Results thereof were shown in FIG. 7A and FIG. 7B (the number of n=3).

### Results

As is clear from the results shown in FIG. 7 and FIG. 7B, in the organ of Example 3 into which the foreign substance was injected by using the method of the present invention, the green dye remained inside the organ, and no leakage of the foreign substance (the green dye) occurred (the number of n=3). It is presumed that the injection mark was filled back to a certain extent by the metanephros mesenchymal cells or the like. In the organ of Comparative Example 1 into which the foreign substance was injected according to the known method described in Non-Patent Document 2, as is clear from the result shown in FIG. 7A, it can be understood that the foreign substance (the green dye) leaked out from an injection hole (the number of n=3). As is clear from the result shown in FIG. 7B, it can be understood that the foreign substance (the green dye) also leaked to the ureter (the number of n=3).

### Example 4

The present inventors have previously produced chimeric pigs each having a donor-derived pancreas by injecting germ cells (donor germ cells) derived from a normal pig into an embryo (a host embryo) derived from a pig that has the Pdx1-Hes1 gene and whose pancreas formation was inhibited (the β cells lacked in the pancreas), and then growing the embryo inside a body of a foster mother (Matsunari et al., PNAS 110:4557-4562 (2013)). It is possible to obtain newborns whose pancreas formation was inhibited (the β cells lacked in the pancreas) at a constant rate by naturally mating male chimeric pigs with wild type female pigs. The newborn pigs used in Example 4 whose pancreas formation was inhibited (the β cells lacked in the pancreas) were obtained by natural mating with such chimeric pigs (see JP 2019-62929A).

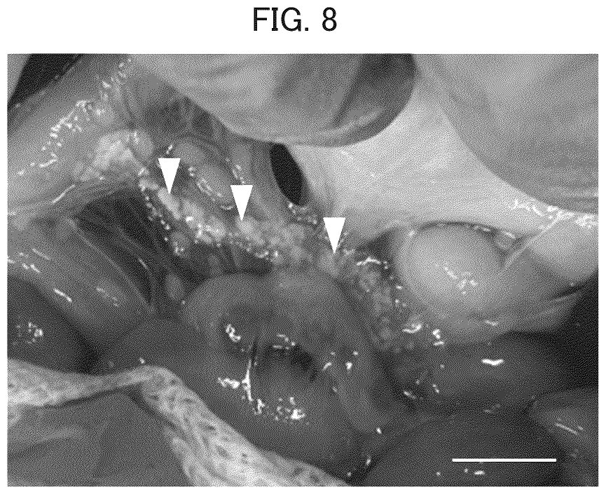

FIG. 8 is a diagram illustrating pancreas tissues obtained by performing a laparotomy on one of the newborn pigs whose pancreas formation was inhibited (the α cells lacked in the pancreas). The scale bar in FIG. 8 is 1 cm. As is clear from FIG. 8, it can be understood that unlike a normal pancreas, a plurality of fragmentary (a major axis of several millimeters) pancreas tissues (arrowheads) were formed. As described above, it can be understood that since the pancreas tissues are very small, it is difficult to inject the foreign substance via the capillary tube in a state in which the pancreas tissues are attached to the pig's body.

As in Example 1, the plurality of pancreas tissue fragments (arrowheads) were cut and semi-embedded in the gelatin gel, and then were placed and positioned on a 35 mm dish (manufactured by Iwaki Co., Ltd.). A capillary tube sucking human iPS cell-derived pancreatic progenitor cells was punctured into each of the plurality of semi-embedded pancreas tissues, and the human iPS cell-derived pancreatic progenitor cells were injected as the foreign substance.

### Results

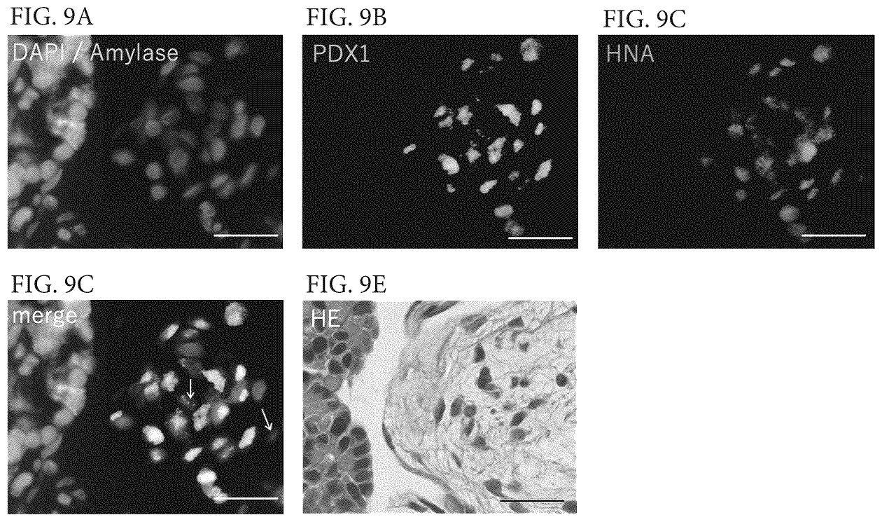

FIGS. 9A-FIG. 9E diagramS illustrating a state of a human iPS cell-derived pancreatic progenitor cell mass injected into one of the plurality of pancreas tissue fragments illustrated in FIG. 8. The scale bar in FIGS. 9A-FIG. 9E is 50 μm. FIG. 9A is a diagram illustrating positions of cell nuclei in the entire tissue by DAPI (4′,6-diamidino-2-phenylindole) staining and a fluorescent immunostaining result of an amylase. As is clear from FIG. 9A, amylase-positive cells was detected, and it can be said that the amylase-positive tissue is the pancreas tissue of the pig. Further, FIG. 9B is a diagram illustrating a fluorescent immunostaining result of PDX1 expression green fluorescence-labeled secondary antibodies. Further, FIG. 9C is a diagram illustrating a fluorescent immunostaining result of human cell nuclei expressing human nuclear antigens (HNAs). As is clear from FIG. 9B and

FIG. 9C, cells that are positive for both PDX1 and HNAs were detected, since the cells are positive for the above, the cells are the “human iPS cell-derived pancreatic progenitor cells.” FIG. 9D is a diagram illustrating a result of merging the respective fluorescent detections described above. FIG. 9E is a diagram illustrating confirmation results of a pancreatic cell structure and a pancreatic tissue structure by hematoxylin-eosin (HE) staining. As is clear from FIG. 9D and FIG. 9E, features of the human iPS cell-derived pancreatic progenitor cells were detected in a pig amylase-positive pancreas cell tissue in which the cells, that is, the β cells lacked, and it can be said that the human iPS cell-derived pancreatic progenitor cells were injected in the tissue. As is clear from the results, it can be understood that the foreign substance (the human iPS cell-derived pancreatic progenitor cells) could be easily injected into the desired injection target zone (the pancreas tissue fragments) of the organ removed from the animal body

1. A method for producing a transplant material including an organ into which a foreign substance is injected, comprising:
bringing at least a portion of an organ removed from an animal body into contact with a member having plasticity and positioning the organ; and
injecting a foreign substance into the positioned organ.

2. The method according to claim 1, wherein the injection is performed from a surface immediately above or near an injection target zone in the organ.

3. The method according to claim 1, wherein the animal body is a fetus body.

4. The method according to claim 1, wherein the organ is an organ for xenotransplantation.

5. The method according to claim 1, wherein the organ is an organ subjected to vitrification freezing after being removed.

6. The method according to claim 5, wherein the member having plasticity is a member having plasticity covering the organ during the vitrification freezing.

7. The method according to claim 1, wherein at least a portion of the organ is embedded in the member having plasticity.

8. The method according to claim 1, wherein the member having plasticity is a gel.

9. The method according to claim 1, wherein a needle-shaped member is used for the injection.

10. The method according to claim 9, wherein a path through which the needle-shaped member passes has a dense structure that is difficult for a liquid to penetrate.

11. The method according to claim 1, wherein the foreign substance refers to a cell derived from an animal other than the animal of the animal body.

12. The method according to claim 1, wherein the organ is a kidney, and the injection target zone is a nephrogenic zone of a metanephros.

13. A transplant material comprising: an organ removed from an animal; and a foreign substance injected into the organ, wherein an injection mark is formed on the organ on a surface immediately above or a surface near a zone in which the foreign substance is injected, the surface immediately above the zone is a surface closest to the zone on a surface of the organ, and
the surface near the zone is a surface present on a side same as the zone with respect to a center of the organ.

14. The material according to claim 13, wherein the foreign substance refers to a cell derived from a portion other than the organ.

15. The material according to claim 13, wherein the surface near the zone refers to a surface including the surface immediately above the zone.

16. The material according to claim 13, wherein the material is used for xenotransplantation.

17. The material according to claim 13, wherein the animal refers to a pig.

18. The material according to claim 13, wherein the material is subjected to vitrification freezing.

19. The material according to claim 13, wherein at least a portion of the injection mark is covered with a gel.

20. A method for transplanting a transplant material, comprising:
transplanting the material according to claim 13 into a recipient.
